SEQUENCE LISTING (4 sequences)

SEQ ID NO: 1 - DNA, 20 bp, Artificial Sequence
  Synthetic forward primer
atggccgttcttagttggtg

SEQ ID NO: 2 - DNA, 20 bp, Artificial Sequence
  Synthetic reverse primer
gaacgccacttgtccctcta

SEQ ID NO: 3 - DNA, 21 bp, Artificial Sequence
  Synthetic forward primer
gcaacaattcctggcgttacc

SEQ ID NO: 4 - DNA, 21 bp, Artificial Sequence
  Synthetic reverse primer
cgaaagccctgtattccgtct

(19) United States
(12) Patent Application Publication
(10) Pub. No.: US 2022/0249511 A1
(43) Pub. Date: Aug. 11, 2022

(54) METHODS FOR THE TREATMENT OF KELOID, HYPERTROPHIC SCARS AND/OR HYPERPIGMENTATION DISORDERS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ DE PARIS, Paris (FR); ASSISTANCE PUBLIQUE-HÔPITAUX DE PARIS (APHP), Paris (FR)

(72) Inventor: Guillaume CANAUD, Paris (FR)

(21) Appl. No.: 17/599,210

(22) PCT Filed: Mar. 27, 2020
(86) PCT No.: PCT/EP2020/058684

§ 371 Date: Sep. 28, 2021

(30) Foreign Application Priority Data
Mar. 29, 2019 (EP) 19305416.0

(51) Int. Cl. A61K 31/553; A61P 17/02; A61K 31/4439; A61K 31/436

(52) U.S. Cl. CPC A61K 31/553; A61K 31/436; A61K 31/4439; A61P 17/02

(57) ABSTRACT

The invention relates to a PI3K inhibitor for use in the treatment of keloid, hypertrophic, bum scars and/or hyperpigmentation disorders in a subject in need thereof. 19 patients underwent multiple surgeries before receiving BYL719. These surgery procedures led to a certain number of severe hypertrophic and keloid scars. Inventors observed that during treatment with oral BYL719, the length, the area and the thickness of the different scars were dramatically reducing without additional treatment. Furthermore, they observed a spontaneous that the nevi were spontaneously decolorating with BYL719. Nevus color was evaluated using an arbitrary visual scale ranging from (5: dark color to 1: normal skin color). This new approach with either BYL719 alone or in combination with other therapeutics seem to be very promising in patients with keloid, hypertrophic, and/or burn scars.

### FIELD OF THE INVENTION

The invention is in the field of dermatology. More particularly, the invention relates to methods and compositions for the treatment of keloid, hypertrophic, burn scars and/or hyperpigmentation disorders.

### BACKGROUND OF THE INVENTION

A scar is an area of fibrous tissue that replaces normal skin after an injury. Scars result from the biological process of wound repair in the skin, as well as in other organs and tissues of the body.

Wound healing is a sophisticated dynamic process that leads to tissue repair or regeneration and has three main time dependent phases: inflammatory phase, proliferative phase and remodeling phase. The healing process starts immediately after skin injury and takes months to complete¹. During the inflammatory phase, many cytokine mediators are activated leading to the recruitment of inflammatory cells, epithelial cells, and fibroblasts. In the second stage of the wound healing, proliferation begins around day 4 or 5 with the relocation of fibroblasts into the wound matrix and inward migration or epithelialization of keratinocytes from the wound margin or hair follicles. The third and last stage in wound healing is the remodeling phase, which usually begins three weeks after tissue injury; this phase is responsible for intra- and interpersonal differences in scar qualities.

Thus, scarring is a natural part of the healing process. Scar tissue is composed of the same protein (collagen) as the tissue that it replaces, but the fiber composition of the protein is different; instead of a random basket weave formation of the collagen fibers found in normal tissue, in fibrosis the collagen cross-links and forms a pronounced alignment in a single direction. This collagen scar tissue alignment is usually of inferior functional quality to the normal collagen randomized alignment.

Usually scars are well delimited. However, in some conditions, fibroblasts and myofibroblasts overproduce collagen (type I and III) leading to hypertrophic scars². These hypertrophic scars are restricted to the original wound area. Importantly, under certain circumstances the scars can grow outside from the original injured area, invading adjacent dermal tissue due to extensive production of extracellular matrix, especially collagen (type I and III), which caused by over expression of cytokines and growth factors. These scars are called keloid. Keloid scars are seen 15 times more frequently in people of sub-Saharan African descent than in people of European descent.

Successful keloid (or hypertrophic scars) treatment is extremely limited. Current treatments are based on surgery, Pressure Garments Therapy, Silicone Gel Sheeting, Onion Extract and Heparin Gel, local corticosteroids, 5-Fluorouracil, bleomycin, Mitomycin C or surgery².

Overgrowth syndromes are rare genetic disorders defined by tissue hypertrophy that can be either localized or generalized. In most cases, the mutations are not inherited but occur during embryogenesis, leading to somatic mosaicism³. The genes involved in overgrowth syndromes are not well characterized but most appear to be part of the PIK3CA/AKT/mTOR pathway⁴⁻⁸, a major actor in cell growth and proliferation⁹. Among the different genes, a gain-of-function mutation of PIK3CA (phosphatidylinositol-3-kinases class Ia, also called p110α) seems to have a prominent role¹⁰. The clinical presentation of patients with PIK3CA gain-of-function mutations is extremely broad owing to not only mosaicism but also the tissue involved¹¹. Patients usually have complex tissue malformations, including abnormal vessels, anarchic adipose tissue, muscle hypertrophy and/or bone deformation¹²⁻¹⁶. Due to the wide variability of clinical presentation and the difficulty of genetic identification, which most often requires a biopsy of the affected area, the exact prevalence of PIK3CA gain-of-function mutations is yet unknown. Since 2014, patients with overgrowth syndrome harboring a PIK3CA mutation have been included in the PIK3CA-related overgrowth spectrum (or syndrome) (PROS) group¹².

Accordingly, there is a need to find new strategies to treat keloid, hypertrophic scars and/or hyperpigmentation disorders.

### SUMMARY OF THE INVENTION

The present invention relates to a PI3K inhibitor for use in the treatment of keloid, hypertrophic scars and/or hyperpigmentation disorders in a subject in need thereof. In particular, the present invention is defined by the claims.

### DETAILED DESCRIPTION OF THE INVENTION

Inventors recently reported the outcome of patients with a rare genetic condition called PIK3CA-Related Overgrowth Syndrome (PROS) treated with a PIK3CA inhibitor called BYL7193. PROS are the consequence of a post-zygotic mutation that occurs in only a few cells during early developments, resulting in mosaic expression of mutant PI3KCA with various patient phenotypes. The phenotypes are ranging from isolated macrodactyly to congenital lipomatous overgrowth with vascular epidermal and skeletal anomalies (CLOVES) syndrome. The phosphoinositide-3-kinases (PI3Ks) are key lipid kinases that control signaling pathways involved in cell proliferation, motility, survival and metabolism.

Interestingly, most of their 19 patients underwent multiple surgeries before receiving BYL719. These surgery procedures led to a certain number of severe hypertrophic and keloid scars.

Inventors observed that during treatment with oral BYL719, the length, the area and the thickness of the different scars were dramatically reducing without additional treatment. They also observed a reduction in size of a burn scar.

Furthermore, they observed that the nevi were spontaneously decolorating with BYL719. Nevus color was evaluated using an arbitrary visual scale ranging from (5: dark color to 1: normal skin color).

This new approach with either BYL719 alone or in combination with other therapeutics seem to be very promising in patients with keloid, hypertrophic, burn scars and/or nevus coloration.

PI3K Inhibitor for Use in the Treatment of Keloid, Hypertrophic Scars and/or Hyperpigmentation Disorders

Accordingly, in a first aspect, the present invention relates to a PI3K inhibitor for use in the treatment of keloid, hypertrophic, burn scars and/or hyperpigmentation disorders in a subject in need thereof.

In a particular embodiment, the invention relates to a method for inducing nevus decoloration in a subject in need thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the term “keloid” refers to an excessive accumulation of extracellular matrix proteins, leading to an overabundance of collagen formation. Abnormal skin scarring can occur, post-injury in genetically susceptible individuals. As used herein, the term “keloid scars” refers to an excessive scar in which the dense fibrous tissue extends beyond the borders of the original wound or incision, and does not usually regress spontaneously.

As used herein the term “hypertrophic scars” refers to an overgrowth of dense fibrous tissue that result from abnormal wound healing. In contrast to keloids, hypertrophic scars do not extend beyond the original boundaries of a wound. Also, unlike keloids, hypertrophic scars typically reach a certain size and then stabilize or regress.

As used herein the term “burn scars” refers to damaged skin that result from exposure to the heat, certain chemicals or even electricity. The severity of the burn depend on how long a person is exposed to the heat as well as its intensity. First-degree burns damage the outer layer of the skin (the epidermis) and cause redness and pain. Second-degree burns affect both the epidermis and the layer under the skin (the dermis). As well as pain and redness, people with second-degree burns may experience blisters. Third-degree burns are the most severe. They damage the top two layers of skin but may also damage the bones and tendons and can affect nerve endings. People with third-degree burns may also notice their skin turn white or black.

As used herein the terms “hyperpigmentation disorders” or “hyperpigmentary skin disorder” are used interchangeably and refer to the darkening of an area of skin or nails caused by increased melanin. Hyperpigmentation is the result of either of two occurrences: (1) an abnormally high concentration of melanocytes produce melanin or (2) when melanocytes are hyperactive. Hyperpigmentation disorders are can affect any part of the body including the face, hands, and neck. Hyperpigmentation disorder is selected form the group consisting of but not limited to solar lentigines, melasma, freckles, age spots, post-acne pigmentation and post-inflammatory hyperpigmentation. The term “lentigo/lentigenes” or “solar lentigines,” also known as a sun-induced freckle or senile lentigo, is a dark (hyperpigmented) lesion caused by natural or artificial ultraviolet (UV) light. The term “melasma” also called as pregnancy-induced melasma. It is also known as pregnancy mask or chloasma. With melasma, the pigmentation is generally symmetrical and has clearly defined edges. The term “freckles” refers to flat circular spots which are usually tan or light brown in colour. While freckles are an extremely common type of hyperpigmentation, they are more often seen among people with a lighter skin tone. The term “age spots” refers to tan, brown or black in colour. Age spots are oval in shape and the size varies from freckle size to more than 13 mm It is also known as liver spots and they tend to develop on the face and other photo-exposed areas after the age of 40. The term “post acne pigmentation” refers to marks caused by acne. They can be observed in more than 60% of acne in some ethnies. In most cases pigmentary marks which are dark in colour result from an overproduction of melanin in reaction to skin inflammation at the affected area. Without proper treatment, post-acne pigmentation may take months or even years to fade off. The term “post inflammatory hyperpigmentation” refers to the marks caused by an injury or inflammation to the skin, there is an increased production of colour pigment in such conditions. More particularly, the present invention allows to treat nevus coloration and giant pigmented nevus.

As used herein the term “nevus coloration” also known as mole, birthmark, and beauty mark, refers to a visible, circumscribed, chronic lesion of the skin or mucosa. The term “nevus cells” refers to a development of normal cells forming pigment melanocytes. Melanocytic nevi are present in almost every human being in a number, size and intensity of different color. Nevi may be located at skin level or protruding above the skin level (spherical, pedunculated or superimposed), dot-shaped or large surface warty, bulging or smooth pigmented lesions, and coloration is color the skin to brown and black. The number of acquired melanocytic nevi increases throughout life.

As used herein, the term “giant pigmented nevus” refers to a large, or giant, congenital melanocytic nevus (LCMN or GCMN) which is a pigmented skin lesion of more than 20 cm—or 40 cm respectively, projected adult diameter, composed of melanocytes, and presenting with an elevated risk of malignant transformation.

As used herein, the term “inducing process of nevus decoloration” refers to a process which allows to accelerate the decoloration of a nevus in a subject.

As used herein, the term “subject” refers to any mammals, such as a rodent, a feline, a canine, and a primate. Particularly, in the present invention, the subject is a human afflicted with or susceptible to be afflicted with keloid. In particular embodiment, the subject is a human afflicted with or susceptible to be afflicted with hypertrophic scars. In another embodiment, the subject is a human with or susceptible to be afflicted with a hyperpigmentation disorder. In another embodiment, the subject is a human with or susceptible to be afflicted with nevus coloration.

As used herein, the term “PI3K” refers to phosphoinositide 3-kinases also called phophatidylinositide 3-kinases. PI3K belongs to a family of enzymes which phosphorylate the 3′hydroxyl group of the onositol ring of the phosphatidylinositol (PtdIns). The PI3K signalling pathway can be activated, resulting in the synthesis of PIP3 from PIP2. The PI3K family is divided into four different classes: Class I, Class II, Class III, and Class IV. PI3K is involved in the control multiple cellular processes including metabolism, motility, proliferation, growth, and survival, is one of the most frequently dysregulated pathways in human cancers.

As used herein, the term “PI3K inhibitor” refers to a natural or synthetic compound that has a biological effect to inhibit the activity or the expression of PI3K. More particularly, such compound is capable of inhibiting the kinase activity of at least one member of PI3K family, for example, at least a member of Class I PI3K. In particular embodiment, said PI3K inhibitor may be a pan-inhibitor of Class I PI3K (known as p110) or isoform specific of Class I PI3K isoforms (among the four types of isoforms, p110α, p110β, p110γ or p110δ).

In the context of the invention, the PI3K inhibitor refers to an inhibitor of all class of PI3K and isoforms.

In a particular embodiment, the PI3K inhibitor is a peptide, petptidomimetic, small organic molecule, antibody, aptamers, siRNA or antisense oligonucleotide. The term “peptidomimetic” refers to a small protein-like chain designed to mimic a peptide. In a particular embodiment, the inhibitor of PI3K is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the PI3K inhibitor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the PI3K inhibitor is a small molecule which is an isoform-selective inhibitor of PI3K selected among the following compounds: BYL719 (Alpelisib, Novartis), GDC-0032 (Taselisib, Genentech/Roche), BKM120 (Buparlisib), INK1117 (Millenium), A66 (University of Auckland), GSK260301 (Glaxosmithkline), KIN-193 (Astra-Zeneca), TGX221 (Monash University), TG1202, CAL101 (Idelalisib, Gilead Sciences), GS-9820 (Gilead Sciences), AMG319 (Amgen), IC87114 (Icos Corporation), BAY80-6946 (Copanlisib, Bayer Healthcare), GDC0941 (Pictlisib, Genentech), IPI145 (Duvelisib, Infinity), SAR405 (Sanofi), PX-866 (Oncothyreon) or their pharmaceutically acceptable salts. Such PI3K inhibitors are well-known in the art and described for example in Wang et al Acta Pharmacological Sinica (2015) 36: 1170-1176.

In a particular embodiment, the PI3K inhibitor is BYL719. As used herein, the term “BYL719” is an ATP-competitive oral PI3K inhibitor selective for the p110α isoform that is activated by a mutant PIK3CA gene (Furet P., et al. 2013; Fritsch C., et al 2014). This molecule is also called Alpelisib and has the following formula and structure in the art C₁₉H₂₂F₃N₅O₂S:

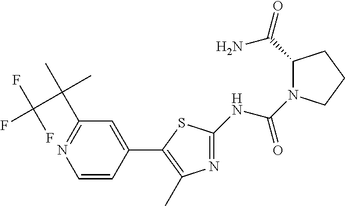 [SMILES: Cc1nc(NC(=O)N2CCC[C@H]2C(N)=O)sc1-c1ccnc(C(C)(C)C(F)(F)F)c1]

In a particular embodiment, the PI3K inhibitor is GDC-0032, developed by Roche. This molecule also called Taselisib has the following formula and structure in the art C₂₄H₂₈N₈O₂:

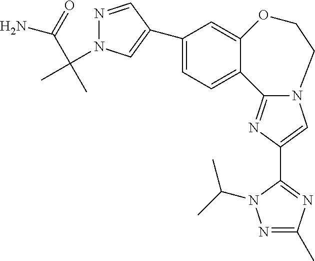 [SMILES: Cc1nc(-c2cn3c(n2)-c2ccc(-c4cnn(C(C)(C)C(N)=O)c4)cc2OCC3)n(C(C)C)n1]

In some embodiments, the PI3K inhibitor is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively);

sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001 ; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the PI3K inhibitor is an intrabody having specificity for PI3K. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In some embodiments, the PI3K inhibitor is a short hairpin RNA (shRNA), a small interfering RNA (siRNA) or an antisense oligonucleotide which inhibits the expression of USP14. In a particular embodiment, the inhibitor of JMY expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PI3K expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339 : 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339 : 823-826), Xenopus tropicalis (Guo et al., 2014, Development, Vol. 141 : 707-714.), yeast (DiCarlo et al., 2013, Nucleic Acids Res., Vol. 41 : 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6 : 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56 : 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiments, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System ” (2015); Cell; 163, 1-13).

In a further embodiment, the PI3K inhibitor is an AKT inhibitor.

As used herein, the term “AKT” refers also known as protein kinase B or PKB is well known in the art and refers to a protein serine/threonine kinase that was first discovered as an oncogene transduced by the acute transforming retrovirus (AKT-8).

As used herein, the term “AKT inhibitor” refers to a compound (natural or synthetic) that inhibits the signalling pathway AKT kinase (also called protein kinase B or PKB). In a particular embodiment, the AKT inhibitors is a peptide, petptidomimetic, small organic molecule, antibody, aptamers, siRNA or antisense oligonucleotide.

Several chemical classes of small-molecule AKT inhibitors with varying potencies and specificities for the different AKT isoforms have now been developed. These include phosphatidylinositol analogs, ATP-competitive small molecules, pseudosubstrate compounds, and allosteric inhibitors.

Exemplary AKT inhibitors that are contemplated by the invention include but are not limited to, for example, those as described in the following international patent applications which are hereby incorporated by reference in their entireties: aminofurazans (WO2005/019190), substituted pyrimidines (WO2008/006040), and substituted pyridines (WO2009/032653).

In a particular embodiment, the AKT inhibitor is selected from the group consisting of Perifosine KRX-0401), XL418, GSK690693, AT13148, A-443654, MK-2206 2HCl, GSK690693, Ipatasertib (GDC-0068), Capivasertib (AZD5363), SC66, PF-04691502, AT7867, Triciribine, CCT128930, A-674563, PHT-427, Miltefosine, Honokiol, TIC10 Analogue, Uprosertib (GSK2141795), TIC10, Akti-½, Miransertib (ARQ 092) HCl, Afuresertib (GSK2110183), AT13148, Deguelin and SC79.

In a particular embodiment, the AKT inhibitor is Miransertib.

In a particular embodiment, the PI3K inhibitor is BYL719, Taselisib or Miransertib.

The PI3K inhibitor as described above is formulated for oral, cutaneous or topical administration.

As used herein the terms “administering” or “administration” refer to the act of injecting or otherwise physically delivering a substance as it exists outside the body (e.g., an inhibitor of PI3K) into the subject, such as by, intravenous, intramuscular, enteral, subcutaneous, parenteral, systemic, local, spinal, nasal, topical or epidermal administration (e.g., by injection or infusion).

In a particular embodiment, the inhibitor of PI3K is formulated as a paste, an ointment, a suspension, a solution or a cream, a gel or a spray.

In a particular embodiment, the inhibitor of PI3K is formulated as is a cream.

Combined Preparation:

In a second aspect, the invention relates to a PI3K inhibitor and an mTOR inhibitor as a combined preparation for use in the treatment of keloid, hypertrophic, burn scars and/or hyperpigmentation disorders.

In a particular embodiment, the PI3K inhibitor for use according to the invention, and a mTOR inhibitor as a combined preparation for simultaneous, separate or sequential use in the treatment of keloid, hypertrophic, burn scars and/or hyperpigmentation disorders in a subject in need thereof.

As used herein, the term “mTOR” refers to mammalian target of rapamycin, kinase that in humans is encoded by the mTOR gene. mTOR is a member of the phosphatidylinositol 3-kinase-related kinase family of protein kinases (PI3K). The naturally occurring human mTOR gene has a nucleotide sequence as shown in Genbank Accession number NM_004958.3 and the naturally occurring human mTOR protein has an aminoacid sequence as shown in Genbank Accession number NP_004949.1. The murine nucleotide and amino acid sequences have also been described (Genbank Accession numbers NM_020009.2 and NP_064393.2). mTOR is involved in different pathways, including insulin, growth factors (such as IGF-1 and IGF-2), and amino acids, cellular nutrient, oxygen, and energy levels.

The term “mTOR inhibitors” refers to a class of drugs that inhibit mTOR. mTOR inhibitors inhibits cellular metabolism, growth, proliferation, and the formation and signaling through two protein complexes, mTORC1 and mTORC2. mTOR inhibitors are well known in the art. In the context of the invention, mTOR inhibitor is selected from the group consisting of rapamycin and rapalogs (sirolimus; temsirolimus; everolimus; deforolimus); vincristine; dactolisib or BEZ235 (phase I/II of clinical trial; Novartis); or sapanisertib (phase II of clinical trial; NCI).

In a particular embodiment, the mTOR inhibitor is rapamycin.

In a particular embodiment, the mTOR inhibitor is everolimus.

In a particular embodiment, the PI3K inhibitor for use according to the invention and, a mTOR inhibitor, as a combined preparation for simultaneous, separate or sequential use in the treatment of methods for the treatment of keloid, hypertrophic, burn scars and/or hyperpigmentation disorders in a subject in need thereof, wherein the PI3K inhibitor is BYL719 and the mTOR inhibitor is rapamycin.

In a particular embodiment, the PI3K inhibitor for use according to the invention and, a mTOR inhibitor, as a combined preparation for simultaneous, separate or sequential use in the treatment of methods for the treatment of keloid, hypertrophic, burn scars and/or hyperpigmentation disorders in a subject in need thereof, wherein the PI3K inhibitor is BYL719 and the mTOR inhibitor is everolimus.

The PI3K and/or mTOR inhibitors as described above can be used as part of a multi-therapy for the treatment of keloid, hypertrophic, burn scars and/or nevus coloration in a subject in need thereof.

The PI3K and/or mTOR inhibitors as described above can be used as part of a multi-therapy for inducing nevus decoloration.

The PI3K inhibitor can be used alone as a single inhibitor or in combination with other inhibitors like mTOR inhibitors. When several inhibitors are used, a mixture of inhibitors is obtained. In the case of multi-therapy (for example, bi-, tri- or quadritherapy), at least on other inhibitor can accompany the PI3K inhibitor.

In a particular embodiment, the PI3K and mTOR inhibitors can be combined as a bi-therapy for use in the treatment of keloid, hypertrophic, burn scars and/or hyperpigmentation disorders.

In a particular embodiment, the PI3K and mTOR inhibitors can be combined as a bi-therapy for use in the inducing process of nevus decoloration.

In a particular embodiment, the PI3K and mTOR inhibitors can be combined for use as a bi-therapy, wherein the PI3K and mTOR inhibitors are BYL719 and rapamycin respectfully.

In a particular embodiment, the PI3K and mTOR inhibitors can be combined for use as a bi-therapy, wherein the PI3K and mTOR inhibitors are BYL719 and everolimus respectfully.

In a particular embodiment, the PI3K, the AKT and mTOR inhibitors can be combined as tri-therapy for use in the treatment of keloid, hypertrophic, burn scars and/or hyperpigmentation disorders.

In a particular embodiment, the PI3K, the AKT and mTOR inhibitors can be combined as tri-therapy for use in the inducing process of nevus decoloration.

The present invention also relates to a method for treating keloid, hypertrophic, burn scars and/or hyperpigmentation disorders in a subject in need thereof comprising a step of administering the subject with a therapeutically effective amount of a PI3K inhibitor.

In a further embodiment, the invention relates to a method for inducing nevus decoloration in a subject in need thereof comprising a step of administering the subject with a therapeutically effective amount of a PI3K inhibitor.

In a particular embodiment, the method according to the invention, wherein the PI3K inhibitor and/or a mTOR inhibitor, as combined preparation for use simultaneously, separately or sequentially in the treatment of keloid, hypertrophic, burn scars and/or hyperpigmentation disorders.

In a particular embodiment, the method according to the invention, wherein the PI3K inhibitor and/or a mTOR inhibitor, as combined preparation for use simultaneously, separately or sequentially in the inducing process of nevus decoloration.

As used herein the terms “administering” or “administration” refer to the act of injecting or otherwise physically delivering a substance as it exists outside the body (e.g., an inhibitor of PI3K alone or in a combination with a mTOR inhibitor) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a particular embodiment, the inhibitor of PI3K alone or in a combination with an mTOR inhibitor are administered topically to the subject suffering or susceptible to suffer from keloid, hypertrophic, burn scars and/or hyperpigmentation disorders.

In a particular embodiment, the inhibitor of PI3K alone or in a combination with an mTOR inhibitor are administered topically to the subject who needs nevus decoloration.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Cosmetic Application

In another aspect, the present invention relates to a PI3K inhibitor suitable for cosmetic application in a subject in need thereof.

In a particular embodiment, the invention relates to the cosmetic use of PI3K inhibitor for inducing nevus decoloration in a subject in need thereof.

In a particular embodiment, the invention relates to a cosmetic composition for use in the inducing process of nevus decoloration.

In a particular embodiment, the cosmetic composition according to the invention comprises a PI3K inhibitor.

In a particular embodiment, the cosmetic composition according to the invention wherein the PI3K inhibitor is an AKT inhibitor.

In a particular embodiment, the invention relates to a cosmetic composition comprising a PI3K inhibitor and an mTOR inhibitor as described above.

In a particular embodiment, the cosmetic composition according to the invention wherein the PI3K inhibitor and a mTOR inhibitor as combined preparation for use simultaneously, separately or sequentially in the inducing process of nevus decoloration.

The term “cosmetic composition” is intended to mean any cosmetic composition, for example a composition that can be brought into contact with the superficial parts of the human body, for example the epidermis, the hair and capillary systems, the external organs and mucous.

Pharmaceutical Composition

In a third aspect, the invention relates to a pharmaceutical composition for use in the treatment of keloid, hypertrophic, burn scars and/or hyperpigmentation disorders.

In a particular embodiment, the invention relates to a pharmaceutical composition for use in the inducing process of nevus decoloration.

In a particular embodiment, the pharmaceutical composition according to the invention comprises a PI3K inhibitor.

In a particular embodiment, the pharmaceutical composition according to the invention wherein, the PI3K inhibitor is an AKT inhibitor.

In a particular embodiment, the invention relates to a pharmaceutical composition comprising a PI3K inhibitor and an mTOR inhibitor as described above.

In a particular embodiment, the pharmaceutical composition according to the invention wherein the PI3K inhibitor and a mTOR inhibitor as combined preparation for use simultaneously, separately or sequentially in the treatment of keloid, hypertrophic, burn scars and/or hyperpigmentation disorders.

In a particular embodiment, the pharmaceutical composition according to the invention wherein the PI3K inhibitor and a mTOR inhibitor nas combined preparation for use simultaneously, separately or sequentially in the inducing process of nevus decoloration.

In another embodiment, the pharmaceutical composition according to the invention, wherein the PI3K inhibitor is BYL719 (Alpelisib), Taselisib or Miransertib.

The PI3K, mTOR and/or AKT inhibitor as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In some embodiments, the pharmaceutical formulation is suitable for topical administration. In certain embodiments, the present invention provides a topical formulation comprising aPI3K inhibitor.

In a particular embodiment, the present invention provides a topical formulation comprising PI3K and mTOR inhibitors. For example, and not by way of limitation, the present invention provides a topical formulation comprising BYL719. Dosage forms for the topical or transdermal administration of the inhibitors of the present invention include, but are not limited to, powders, sprays, ointments, pastes, creams, lotions, gels, solutions, patches and inhalants In certain non-limiting embodiments, a topical formulation comprises one or more PI3K inhibitor comprised in micelles, liposomes, or non-lipid based microspheres. In certain non-limiting embodiments, such a topical formulation may comprise a permeability enhancing agent such as but not limited to dimethyl sulfoxide, hydrocarbons (for example, alkanes and alkenes), alcohols (for example, glycols and glycerols), acids (for example, fatty acids), amines, amides, esters (for example, isopropyl myristate), surfactants (for example, anionic, cationic, or non-ionic surfactants), terpenes, and lipids (for example, phospholipids).

In a particular embodiment, the formulation is a paste, an ointment, a suspension, a solution or a cream, a gel or a spray. In a particular embodiment, the formulation is a cream.

In a particular embodiment, the dosage form of topical or transdermal administration of the inhibitors of the present invention is a cream.

In certain embodiments, the pharmaceutical formulation can be suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In certain embodiments, the present invention provides a parenteral formulation comprising a PI3K inhibitor and an mTOR inhibitor as a combined preparation. In certain embodiments, the present invention provides a parenteral formulation comprising a PI3K inhibitor and a mTOR inhibitor as a combined preparation. For example, and not by way of limitation, the present invention provides a parenteral formulation comprising BYL719 and an mTOR inhibitor as a combined preparation. In a particular embodiment, when the PI3K inhibitor is combined with a mTOR inhibitor, wherein the combination is formulated for oral, cutaneous or topical use.

Method of Screening

A further object of the present invention relates to a method of screening a drug suitable for the treatment of keloid, hypertrophic, burn scars and/or nevus coloration comprising i) providing a test compound and ii) determining the ability of said test compound to inhibit the activity of PI3K, AKT and/or mTOR.

Any biological assay well known in the art could be suitable for determining the ability of the test compound to inhibit the activity of PI3K, mTOR and/or AKT. In some embodiments, the assay first comprises determining the ability of the test compound to bind to PI3K, AKT and/or mTOR. In some embodiments, a population of cells is then contacted and activated so as to determine the ability of the test compound to inhibit the activity of PI3K, AKT and/or mTOR. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PI3K, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Representative pictures of hypertrophic and keloid scars but also nevus before and during treatment with BYL719 (either 50 mg/day for patients <18 years old or 250 mg/day for patients >18 years old). Quantification of the area changes (percentage) and nevus color change (visual scale). AU: Arbitrary Unit. **P<0.01, ***P<0.001 (one way ANOVA)

FIG. 2: Representative picture of burn scars before and during treatment with BYL719. Quantification of the area (cm2).

FIG. 3: A) Western blot analysis of fibroblasts derived from normal of pathological scars treated with either DMSO, BYL719, taselisib or miransertib. These drugs inhibit the AKT/mTOR pathway. Quantification of P-AKT and P-S6RP in WB. B) Quantification of cells undergoing proliferation while exposed to DMSO, BYL719, taselisib or miransertib. C) Tgfβ mRNA expression in cells exposed to DMSO, BYL719, taselisib or miransertib. D) Ex vivo keloids were cultivated and exposed to either DMSO or BYL719. Western blot analysis demonstrates that BYL719 inhibits the AKT/mTOR pathway. E) BYL719 reduces the proliferation rate in keloids but also F) Tgfβ mRNA expression. AU: Arbitrary Unit. *P<0.05, ***P<0.001 (one-way ANOVA)

### EXAMPLE

Material and Methods

Cohort of PROS Patients Treated with BYL719

The cohort is composed by 19 patients including 15 children (ranging from 4 to 50 years old). Children received 50 mg daily while adults received 250 mg daily. BYL719 was delivered orally every morning with breakfast. Patients were followed at regular intervals:

weekly for 8 weeks, every two weeks for 1 month and then monthly. All patients underwent clinical examination, and areas of overgrowth, nevi as well as visible scars were photographed before and after BYL719 introduction. All pictures were taken by a single operator, always using the same camera (12-megapixel camera), in the same room and at the same distance. Notably, one patient had a large burn scar on the left thigh. Length and surface of the scars were measured using Image J.

Immunofluorescence Studies

Paraffin-embedded kidney sections (4-um) were incubated with anti-P-AKT (Ser473) antibody (Cell Signaling Technology, ref# 4060), anti-P-S6RP antibody (Cell Signaling Technology, ref# 5364), anti-a-smooth muscle cell antibody (Sigma Aldrich, ref# A5228), and anti-KI67 antibody (Thermo Fisher Scientific, ref# RM-9106-51) Immunofluorescence studies were analyzed using a Zeiss LSM 700 confocal microscope.

Cell Culture Experiments

Skin samples (hypertrophic, keloids and burn scars) were collected during surgical procedures. To generate dermal fibroblast cultures, skin samples were minced and incubated at room temperature in 0.05% trypsin-EDTA (ThermoFisher) solution for 30 min with gentle shaking. Cells were collected by centrifuging at 700 g for 10 min, resuspended in cell culture media containing 25% FBS, and plated onto 24 well plates to establish lines. Fibroblast cultures were grown and maintained in 1× MEM (Corning) supplemented with 25% FBS and penicillin/streptomycin (Corning) to a final concentration of 100 IU penicillin and 500 μg.mL-1 streptomycin.

Cells at similar population doubling were plated 1:4 from confluent cultures and allowed to grow until ˜80% confluent. Cells were then starved during 12 hours and exposed during 24 h to either BYL719 (0.1 or 1 μmol.L-1; MedChem Express), taselisib (0.1 or 2. μmol.L-1; MedChem Express) or miransertib (0.1 or 100 2. μmol.L-1) or equal volume of DMSO. We also performed ex vivo keloid scars culture. Keloids were exposed during 24h to either BYL719 (0.1 or 1 μmol.L-1; MedChem Express) or equal volume of DMSO.

Cultures were rinsed with 1X PBS, treated for 10 min with 0.05% Trypsin, collected by centrifugation at 4° C., and pellets were flash frozen on dry ice. Protein lysates were collected by directly adding 1× RIPA buffer containing protease and phosphatase inhibitors. Each experiment was performed in duplicate and repeated at least three times

Western Blot

Western blots were performed as previously described³¹. Briefly, protein extracts from the scars and primary culture of fibroblasts were resolved by SDS-PAGE before being transferred onto the appropriate membrane and incubated with anti-P-AKT (Ser473) antibody (Cell Signaling Technology, ref# 4060), anti-P-S6RP antibody (Cell Signaling Technology, ref# 5364) and anti-GAPDH (Merck Millipore, ref#374), followed by the appropriate peroxidase-conjugated secondary antibody. Chemiluminescence was acquired using a Fusion FX7 camera (Vilbert Lourmat) and densitometry was performed using the Bio1D software (Certain Tech).

RNA Extraction and Real-Time Quantitative PCR

Total RNA was isolated from snap-frozen kidneys using the TRIzol reagent (Sigma) according to the manufacture's protocol. First-strand cDNA was synthesized using the MML-V reverse transcriptase (Promega, USA). Real-time PCR was performed using the iQ-SYBR Green supermix (BioRad) and the iQ5 Multicolor Real-Time PCR Detection System (BioRad) for mRNA detection. 18S rRNA was used as a house keeping gene.

The following primer sequences were used:

18SrRNA:
forward:
(SEQ ID NO: 1)
ATGGCCGTTCTTAGTTGGTG,
reverse:
(SEQ ID NO: 2)
GAACGCCACTTGTCCCTCTA;
TGF-β:
forward:
(SEQ ID NO: 3)
GCAACAATTCCTGGCGTTACC,
reverse:
(SEQ ID NO: 4)
CGAAAGCCCTGTATTCCGTCT.

Data Analysis and Statistics

Data were expressed as the means±s.e.m. Differences between the experimental groups were evaluated using ANOVA, followed when significant (P<0.05) with the Tukey-Kramer test. When only two groups were compared, Mann-Whitney tests were used. The statistical analysis was performed using the Graph Prism software.

Results

BYL719 Improves Hypertrophic, Keloid and Burn Scars in Patients

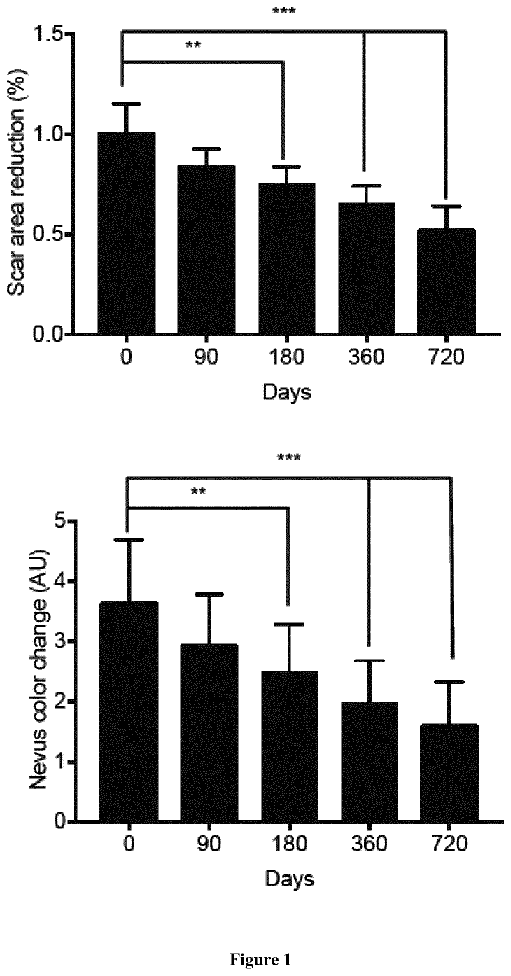

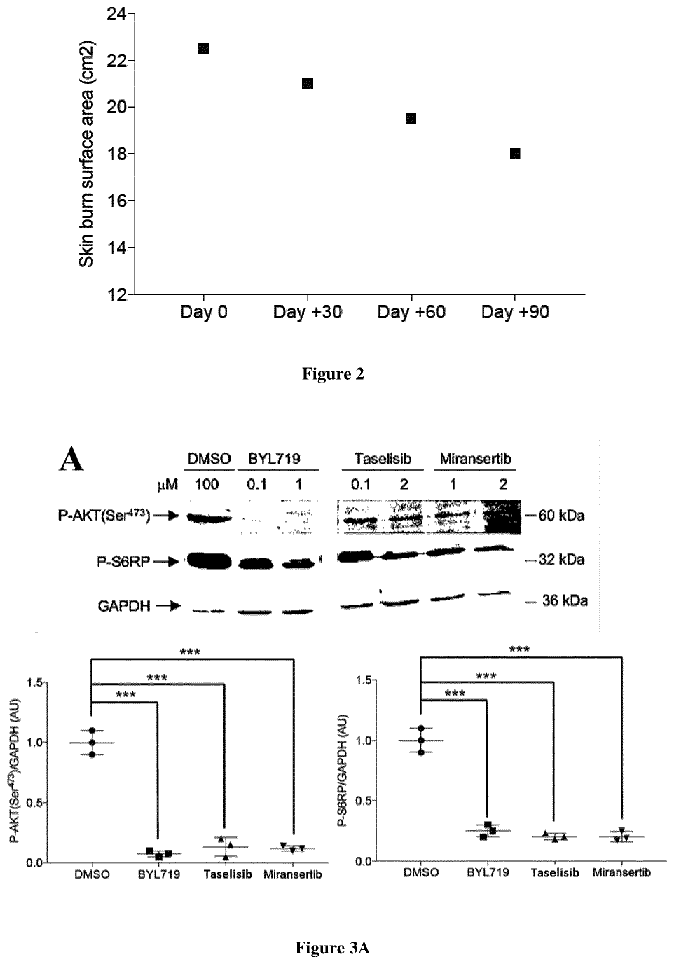

Following the introduction of BYL719 at a daily dose of 250 mg in adults or 50 mg in children, we observed a progressive improvement of the length, the surface area and the thickness of the different hypertrophic and keloids scars. As shown on FIG. 1, inventors have observed that during treatment with oral BYL719, the length, the area and the thickness of the different scars were dramatically reducing without additional treatment. Furthermore, they observed that the nevi were spontaneously decolorating with BYL719. Nevus color was evaluated using an arbitrary visual scale ranging from (5: dark color to 1: normal skin color). Interestingly, we also observed a reduction in size of the burn scar (FIG. 2).

PIK3CA/AKT/mTOR Pathway is Activated in Hypertrophic, Keloid and Burn Scars

We then investigated the activation of the PIK3CA pathway in pathological scars. To this aim, we assessed the phosphorylation status of AKT on the residue Ser473 and S6RP. Both proteins are downstream targets of PIK3CA. Interestingly, whereas AKT and S6RP phosphorylation were not detectable in normal scars, pathological scars (hypertorphic, keloids and burns) demonstrated a high degree of activation associated with a high rate of proliferation determined by the number of KI67 positive cells (data not shown).

PIK3CA/AKT/mTOR Pathway Inhibition in Fibroblasts Derived from Pathological Scars (Hypertrophic, Keloid and Burn) but also Ex vivo Culture of Keloids Improves Proliferation and Fibrosis Synthesis.

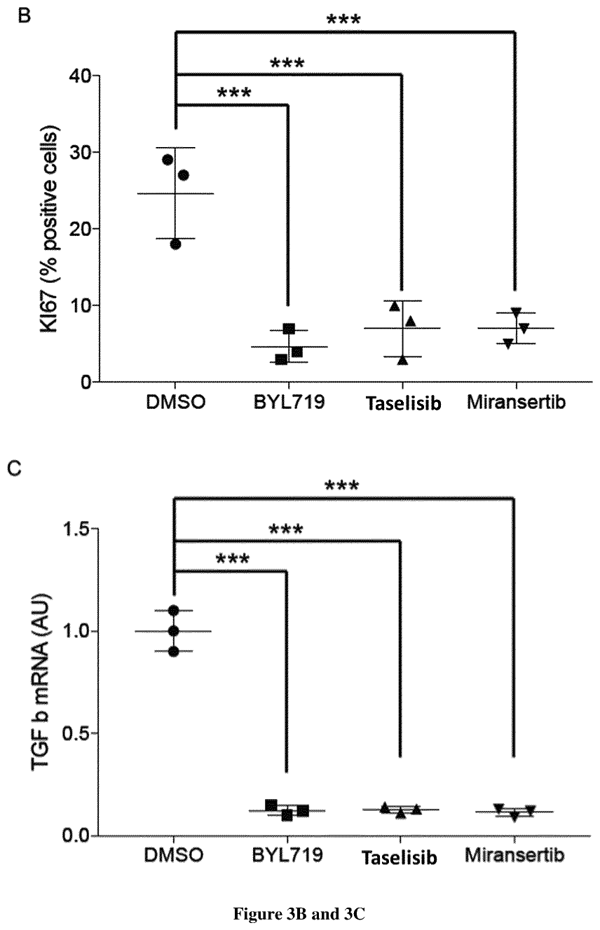

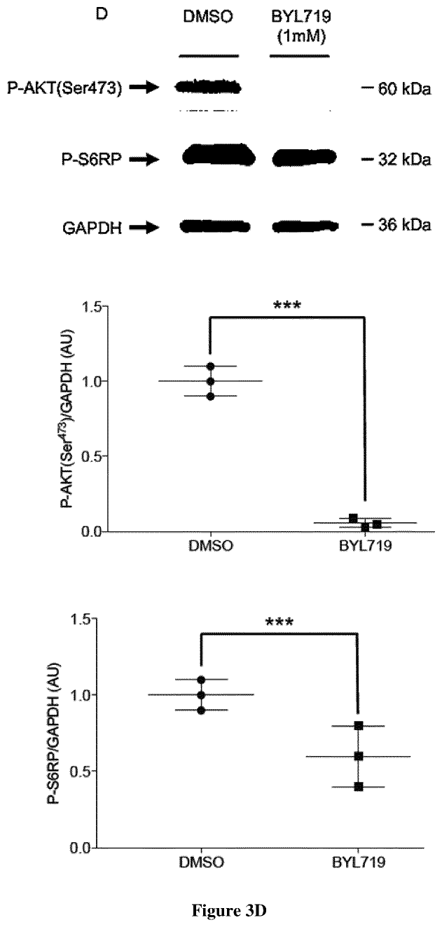

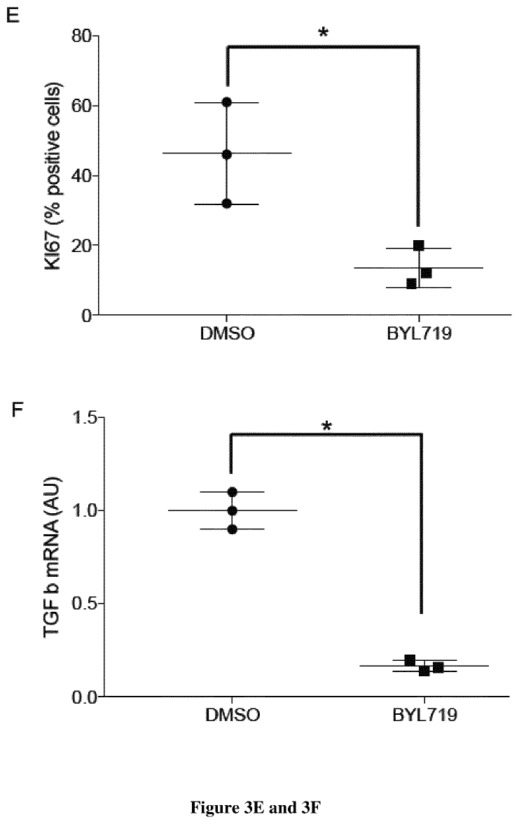

We exposed primary fibroblasts derived from pathological scars to DMSO, BYL719, taselisib (another PIK3CA inhibitor) or miransertib (an AKT inhibitor). As expected, we observed that the different drugs were able to efficiently block the AKT/mTOR pathway (FIG. 3A). Importantly, we noticed that these drugs reduced the proliferation (FIG. 3B) but also the mRNA expression of Tgfβ, a marker of fibrosis (FIG. 3C).

We then exposed in vitro keloid scars removed during surgical procedures to either DMSO or BYL719. We found that BYL719 inhibits the AKT/mTOR pathway (FIG. 3D) but also reduce the proliferation rate (FIG. 3E) and Tgfβ mRNA expression compared to DMSO (FIG. 3F).

This new approach with either BYL719 alone or in combination with other therapeutics seem to be very promising in patients with keloid, keloid, and burn scars and/or hyperpigmentation disorders.

### REFERENCES

Throughout this application, various references describe the state of the art to which this invention pertains. The disclosures of these references are hereby incorporated by reference into the present disclosure.

Mari, W. et al. Novel Insights on Understanding of Keloid Scar: Article Review. J Am Coll Clin Wound Spec 7, 1-7, doi:10.1016/j.jccw.2016.10.001 (2015).

Faguer, S. et al. Rituximab therapy for acute humoral rejection after kidney transplantation. Transplantation 83, 1277-1280, doi:10.1097/01.tp.0000261113.30757.d1 (2007).

Venot, Q. et al. Targeted therapy in patients with PIK3CA-related overgrowth syndrome. Nature 558, 540-546, doi:10.1038/s41586-018-0217-9 (2018).

1. A PI3K inhibitor for use in the treatment of keloid, hypertrophic, burn scars and/or hyperpigmentation disorders in a subject in need thereof.

2. The PI3K inhibitor for use according to claim 1 which is suitable for inducing nevus decoloration in a subject in need thereof.

3. The PI3K inhibitor for use according to claim 1, wherein, the PI3K inhibitor is formulated for oral, cutaneous or topical administration.

4. A PI3K inhibitor and an mTOR inhibitor as a combined preparation for use in the treatment of keloid, hypertrophic, burn scars and/or hyperpigmentation disorders.

5. The PI3K inhibitor and the mTOR inhibitor according to claim 4 as a combined preparation for simultaneous, separate or sequential use in the treatment of keloid, hypertrophic, burn scars and/or nevus coloration in a subject in need thereof.

6. The PI3K inhibitor and the mTOR inhibitor according to claim 4 as a combined preparation for use in inducing nevus decoloration in a subject in need thereof.

7. The PI3K inhibitor for use according to claim 1, wherein, the PI3K inhibitor is BYL719 (Alpelisib), Taselisib or Miransertib.

8. The PI3K inhibitor and the mTOR inhibitor for use according to claim 4 wherein the inhibitors are formulated for oral, cutaneous or topical use.

9. The PI3K inhibitor and the mTOR inhibitor for use according to claim 4, wherein said inhibitors can be used as part of a multi-therapy.

10. A pharmaceutical composition comprising a PI3K inhibitor for use in the treatment of keloid, hypertrophic, burn scars and/or hyperpigmentation disorders in a subject in need thereof.

11. The pharmaceutical composition for use according to claim 10, wherein the pharmaceutical composition further comprises an mTOR inhibitor.

12. The pharmaceutical composition for use according to claim 10, wherein the PI3K inhibitor is BYL719 (Alpelisib), Taselisib or Miransertib.

13. The pharmaceutical composition for use according to claim 10, wherein the pharmaceutical formulation is a paste, an ointment, a suspension, a solution, a cream, a gel or a spray.

14. A method for treating keloid, hypertrophic, burn scars and/or hyperpigmentation disorders in a subject in need thereof comprising administering to the subject a therapeutically effective amount of a PI3K inhibitor.

15. A method of screening a drug suitable for the treatment of keloid, hypertrophic, burn scars and/or hyperpigmentation disorders comprising i) providing a test compound and ii) determining the ability of said test compound to inhibit the activity of PI3K and/or mTOR.
